(19) United States
(12) Patent Application Publication
(10) Pub. No.: US 2009/0009715 A1
(43) Pub. Date: Jan. 8, 2009

(54) Confocal Color Ophthalmoscope

(75) Inventor: Michiel Mensink, Den Haag (NL)

Correspondence Address: MARTIN SAVITZKY, PO BOX 1027, FORT WASHINGTON, PA, 19034-1027, US

(73) Assignee: I-OPTICS BV, Rijswijk (NL)

(21) Appl. No.: 12/168,395

(22) Filed: Jul. 7, 2008

Related U.S. Application Data
(60) Provisional application No. 60/947,958, filed on Jul. 4, 2007.

(51) Int. Cl. A61B 3/12

(52) U.S. Cl. 351/213; 351/221

(57) ABSTRACT

A confocal color opthalmoscope having a line-shaped, incoherent light source. The light emitted from the linear light source is focused and scanned to illuminate a region of a retina of an eye with a line oriented perpendicular to the direction of scan. Light reflected from the retina of eye is collected, descanned and focused onto a line shaped detector. A dispersive element may be used to illuminate a multi-slit plate with different spectral components. The retina may then be scanned with multiple slits, each having a different spectral makeup. Light reflected from the retina may be descanned and imaged back to a multiple linear array photo-detector. One of said slits may be illuminated with infra-red radiation, and the others with visible light. A filter may allow only the infra-red light to reach the retina during an initial alignment mode, allowing non-mydriatic alignment of the opthalmoscope.

### CROSS REFERENCE TO RELATED APPLICATIONS

This application is related to, and claims priority from, U.S. Provisional Patent application No. US60/947,958 filed on Jul. 4, 2007 entitled Confocal Color Opthalmoscope, the contents of which are hereby incorporated by reference.

### FIELD OF THE INVENTION

The present invention relates to the field of confocal imaging devices and in particular to fundus cameras and confocal scanning laser opthalmoscopes, which are used for imaging of the retina and medical diagnosis.

### BACKGROUND OF THE INVENTION

The retina can be imaged with various well-known instruments such as, but not limited to, direct opthalmoscopes, binocular indirect opthalmoscopes and fundus cameras.

In these instruments, a light source typically continuously illuminates the entire field being viewed, and the light reflected by the retina is captured by an image detector or the operator's eye. A significant disadvantage of these methods is that they require a substantial amount of visible light which may be uncomfortable for the patient.

In order to reduce the effect of stray light in the retinal imaging, and to obtain high contrast images with invisible and patient-friendly near IR light, Webb and others devised the Scanning Laser Opthalmoscope (SLO) in the 1980's. Their SLO is described in detail in, for instance, U.S. Pat. Nos. 4,213,678, 4,768,873 and 4,781,453, the entire contents of which are hereby incorporated by reference.

In an SLO, a laser spot is typically scanned across the retina and the reflected light is de-scanned and imaged onto a sensitive single point detector, such as, but not limited to, an avalanche photo-diode. Such a flying spot optical design, which incorporates a confocal detector, greatly reduces stray, and therefore enhances the image s contrast. In addition, this method allows the use of near infra red light for imaging the retina, which may further reduce discomfort to the patient.

In the SLO, each point on the retina is only illuminated very briefly. A very bright source of illumination must, therefore, be used to obtain a sufficient level of exposure despite the short exposure time. A laser source is typically used as they are typically the only sources capable of delivering the required high brightness.

In a typical SLO, a slow ‘vertical’ scanner may scan the laser beam at a frequency of 10-30 frames per second, while a fast ‘horizontal’ scanner may scan the beam at a frequency of 10.000 Hz or higher, typically in a perpendicular direction.

Integrating the fast scanner into the instrument can be a significant challenge when designing or manufacturing a typical SLO. A resonant scanner, and a spinning polygon scanner, are two possible solutions that are typically used to obtain such high scanning frequencies. Both of these scanners are, however, typically bulky, costly and noisy and usually require complicated electronics to obtain adequate synchronization between the scanners and the signal from the detector.

The complexity of a typical SLO s scanning mechanisms may be a significant part of the high cost of SLO s. High costs are probably a significant factor in confining commercial SLO use to research and niche applications. Another factor that may have prevented SLO s from being a standard tool for retinal imaging is their monochromacity that is in marked contrast to the full color images of typical fundus cameras.

A third factor that may have prevented SLO s from being a standard tool for retinal imaging is that they typically produce lower resolution images than typical digital fundus camera. For instance, a typical SLO produces an image that is less than 500*500 pixels, while a typical digital fundus camera produces images that are at least 1000*1000 pixels.

Several approaches to solving the SLO short comings of high costs and monochromacity have been described in the prior art.

Many of these attempts to devise a ‘Color SLO’ employ two or three different lasers to produce a green, red and possibly a blue image, in order to produce color SLO images. The different color images may be recorded sequentially and then carefully aligned before integration into a single color image. However, even small eye movements may make it impossible to obtain precise superposition of the monochromatic images over the full field of view.

An alternative approach is to switch the three lasers on and off very rapidly so that each pixel is illuminated sequentially. Such systems are described in, for instance, Manivannan s U.S. Pat. No. 6,099,127. Although these images do not require post-alignment and can be superposed immediately, they do require carefully synchronization of the photo detector to obtain the three monochromatic images.

All such approaches typically have a fundamental disadvantage. They all typically require prohibitively high levels of retinal exposure. The maximum permissible amount of illumination may already be required to obtain an image of sufficient contrast in each individual color. The total retinal exposure of any three-laser SLO would, therefore, exceed the maximum permissible levels if the different colors are recorded simultaneously. Such over-exposure may only be avoided by sacrificing resolution, i.e., reducing the number of pixels, or by sacrificing image quality, i.e., the amount of light per pixel.

Another problem of the so-called ‘color SLOs’ is that the colors of retinal images made with lasers may look very different from the colors of white light fundus camera images. In a color SLO, the three monochromatic red, blue and green images are superposed to create a combined image that should resemble the color image as produced by a fundus camera. In a fundus camera the retina is illuminated by a white light source, while the reflected light is directed to a red, green and blue image sensor. However, the colors in the images created with separate green, blue and red lasers may appear very different to the colors in an image made with a white light source. This is due, in part, to the fact that lasers emit light within a very narrow spectrum. Therefore, the retina is only ‘probed’ with three small spikes in the spectrum rather than the full white light spectrum.

Recently, laser sources such a femtosecond Ti:Saffire lasers have been created which are capable of generating ‘white (wide bandwidth) light. These lasers could be integrated in an SLO and combined with a blue, red and green detector. In that way a natural color SLO could be created. However due to the high cost of these lasers, such an instrument would probably be prohibitively expensive outside the realm of clinical research.

Finally, all lasers produce coherent light which typically generates laser speckle in images. Laser speckle typically reduces the resolution of images compared to a non-coherent illumination.

### SUMMARY OF THE INVENTION

The present invention is a confocal color opthalmoscope having a line shaped, incoherent light source capable of emitting light from a plurality of points along its length. The light emitted from the linear, incoherent light source is focused and scanned to illuminate a region of a retina of an eye with a line oriented perpendicular to the direction of scan. Light reflected from the retina of eye is collected, descanned and focused onto a line shaped detector, which is sufficiently narrow so as to provide for a confocal aperture.

In one embodiment of the opthalmoscope, a dispersive element is used to illuminate a multi-slit plate with different spectral components of the line shaped, incoherent light source. The retina may then be scanned with multiple slits, each having a different spectral makeup. Light reflected from the retina may be descanned and imaged back to a multiple linear array photo-detector.

In a further embodiment of the opthalmoscope, one of said slits may be illuminated with infra-red radiation, and the others with visible light. A movable filter may allow only the infra-red light to reach the retinal region during an initial alignment mode. In this way, non-mydriatic alignment of the opthalmoscope may be undertaken.

These and other features of the invention will be more fully understood by references to the following drawings.

### BRIEF DESCRIPTION OF THE DRAWINGS

FIG. 1A is a schematic plan view of an exemplary confocal, line scanning opthalmoscope of the present invention.

FIG. 1B is a schematic side view of an exemplary confocal, line scanning opthalmoscope of the present invention.

FIG. 2A is a schematic plan view of a further, exemplary confocal, line scanning opthalmoscope of the present invention.

FIG. 2B is a schematic side view of a further exemplary confocal, line scanning opthalmoscope of the present invention.

FIG. 3A is a schematic plan view of an exemplary confocal, color line scanning opthalmoscope of the present invention.

FIG. 3B is a schematic side view of an exemplary confocal, color line scanning opthalmoscope of the present invention.

FIG. 4A is a schematic plan view of a further exemplary confocal, color line scanning opthalmoscope of the present invention.

FIG. 4B is a schematic side view of a further exemplary confocal, color line scanning opthalmoscope of the present invention.

FIG. 5 shows an alternative embodiment of the illumination system, in which an incandescent wire is used as source of illumination.

FIG. 6 shows an alternative embodiment of the illumination system, in which 4 parallel line-shaped LEDs as built into one component are used as source of illumination.

FIG. 7 shows an alternative embodiment of the illumination system, in which 4 separate-line shaped LEDs are used as source of illumination.

FIG. 8 shows a schematic view of an exemplary confocal color scanning system in use.

### DETAILED DESCRIPTION

The novel Confocal Color Opthalmoscope (CCO) of the present is an affordable and compact retinal camera, capable of recording high resolution and high contrast confocal retinal images that may be used for general retinal diagnostic purposes, for screening purposes or for purposes benefiting from spectroscopy.

Such a camera may create confocal retinal images in color, such that the colors closely resemble the colors in fundus camera images. Such a camera may also allow multi-spectral imaging of the retina in which the images of the retina are made using multiple colors, with each color having a narrow spectral range. Such a camera may also eliminate the need for dilation of the pupil. Dilation is frequently required to prevent constriction of the pupil as a result of the bright light used for imaging the retina in e.g., fundus cameras.

In a preferred embodiment of the confocal, color line scanning opthalmoscope, the illumination may be generated by a source of illumination which is not is laser. This is in contrast with the essential requirement to use a laser light source in prior art confocal cameras, which are all variations of the Scanning Laser Opthalmoscope.

The light source used in the present invention may have a wide spectrum of emission, and emits non-coherent light. Examples of such a sources are, but are not limited to, a high brightness arc lamps, an incandescent lamp, an LEDs or a ‘white light’ laser or some combination thereof.

The brightness of a source is an indication of the number of photons emitted from a surface area of the source. The brightness of non-laser light sources is usually a factor 100-10,000 lower than the brightness of a laser. The brightness of non-laser light sources is normally insufficient for imaging the retina with a traditional flying spot SLO.

In a preferred embodiment of the present invention, the non-laser illumination that scans the retina is shaped as four thin lines of light each line having a different color. This in contrast to the single point of light that was used in prior art confocal cameras.

By scanning a line of light across the retina, wherein the line e.g., contains 500-2000 pixel points, instead of a single point of light, the exposure time per retinal point can be a factor 500-2000 longer. Therefore, the illumination intensity of each point on the line can be a factor 500-2000 lower.

This enable the use of a less bright light source, and permits using other light sources than lasers. Therefore the lower brightness of non-laser light sources may no longer pose a problem.

In a preferred embodiment of the present invention, multiple parallel lines of light, having different colors, are scanned across the retina using a galvanometer scanner and an imaging system. The reflected light returns through the imaging system, is de-scanned, and then transmitted to a detector which consists of multiple parallel lines of detector elements. The accumulated electrons in the detector elements are read out and digitized by an analog to digital conversion means. The digital values are pre-processed and subsequently transferred to a computer using digital transfer means. The computer allows further processing, storage and display of the images.

Alternatively, one line of light may be scanned across the retina and imaged on a line detector while the color of illumination is varied in time.

In 1988 Webb in U.S. Pat. No. 4,768,874, the contents of which are hereby incorporated by reference, showed that scanning a thin line of laser light across the retina, de-scanning the returning light and subsequently imaging this light on a line detector, is a method that still retains much of the confocality of a point scan. Line scan images, thanks to their partial confocality, have a much higher contrast compared to the area-based illumination of a fundus camera.

Further, when scanning a line across the retina instead of a point, only one scanner is required instead of two scanners, and the optical path of the camera system can be much shorter. Therefore optics, electronics and mechanics can be simplified, all of which contribute to a decrease in production costs and a decrease in size.

Another advantage of the present invention is that light from a non-laser source is not coherent. Therefore speckles, which plague the laser-based SLO systems, are absent, and therefore the images of the CCO present a higher resolution.

In a preferred embodiment of the present invention, an bright arc lamp illuminates an assembly of filters or dispersive elements, lenses and parallel slits in order to create four very narrow parallel lines of light. Each line of light may be composed of a small or broad spectrum of light as determined by the use of one or more filters. By using three broad spectrum lines of light centered on blue, green and red, the white light conditions of a fundus camera may be replicated in such a system. A fourth line of light may contain near infra red light for patient friendly, non-mydriatic focusing and alignment. By using one or more lines with a narrow spectrum light, spectroscopic measurements may be performed.

In a preferred embodiment of the invention, one or more LEDs, in which the emissive areas are shaped like a thin line, may be used to create the lines of incoherent light.

In yet another embodiment, a long and thin incandescent wire may be used as the source of illumination. An assembly of filters, prisms, lenses and slits may be used to create the four thin lines of illumination.

In accordance with yet another aspect of the invention, at first only infra-red illumination may be used for proper alignment and focusing. Once the patient's eye is properly aligned and in focus, the visible light illumination may be switched on. At least five frames may typically be acquired before the pupil contracts. A series of high quality, color images may, therefore, be created without dilating the eye pupil.

In yet another aspect of the invention, the information gathered with the different colors of illumination is used for qualitative indication or even quantitative measurement of certain properties of the retina; for instance the concentration of oxygenized hemoglobin can be measured in retinal blood vessels, the glucose concentration, as well as concentration levels of other molecules.

In a preferred embodiment of the invention, the image sensor may be a quad-linear or tri-linear array.

In another embodiment of the invention, an area sensor, such as CCD or CMOS may be employed with appropriate electronics that may read out only selected lines of photosensitive elements. The confocality of such a system may, for instance, be decreased by using multiple adjacent lines of elements instead of one, for each color.

In yet another embodiment of the invention, a single linear array sensor is employed, which is illuminated sequentially by different colors of illumination.

In a preferred embodiment of the invention, the imaging system may be achromatic and uses several aspherical or spherical focusing mirrors. In another embodiment of the invention, the imaging system may use lenses.

A preferred embodiment of the invention will now be described in detail by reference to the accompanying drawings in which, as far as possible, like elements are designated by like numbers.

Although every reasonable attempt is made in the accompanying drawings to represent the various elements of the embodiments in relative scale, it is not always possible to do so with the limitations of two-dimensional paper. Accordingly, in order to properly represent the relationships of various features among each other in the depicted embodiments and to properly demonstrate the invention in a reasonably simplified fashion, it is necessary at times to deviate from absolute scale in the attached drawings. However, one of ordinary skill in the art would fully appreciate and acknowledge any such scale deviations as not limiting the enablement of the disclosed embodiments.

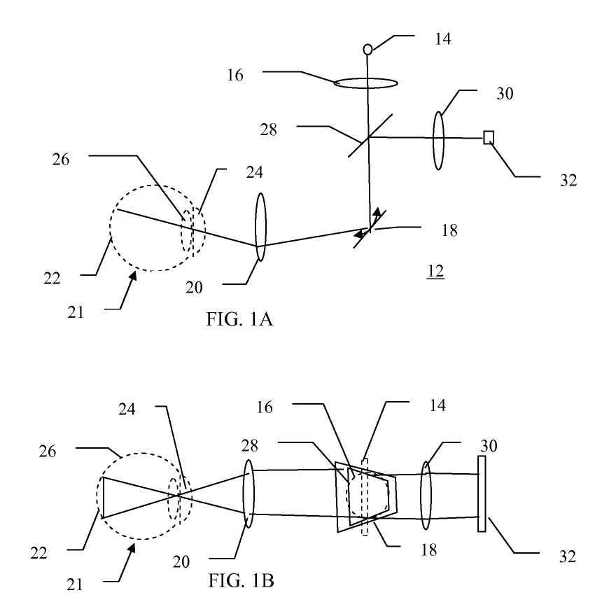

FIG. 1A is a schematic plan view of an exemplary confocal, line scanning opthalmoscope 12 of the present invention. The exemplary confocal, line scanning opthalmoscope 12 consists of a line shaped, incoherent light source 14, a first optical element 16, a scanning element 18, a second optical element 20, a beam-splitter 28, a third optical element 30 and a detector 32.

The line shaped, incoherent light source 14 may be, but is not limited to, a heated incandescent wire emitting light from a plurality of points along its length, an arc lamp emitting a bright white light in the gap between anode and cathode, a high brightness white light emitting diode (LED), including a custom line shaped LED, or a white light laser such as, but not limited to, a Ti-Saphire femto-second laser, or some combination thereof.

The first optical element 16 may be, but is not limited to, a lens, a combination of lenses, an achromatic lens, a holographic imaging element or a mirror, an aspheric mirror or some combination thereof and may be coated with suitable anti-reflection coatings.

The scanning element 18 may be, but is not limited to, a back-and-forth scanning mirror driven by a galvanometer motor such as, but not limited to, the model 6220H moving magnet closed loop galvanometer based optical scanner as supplied by Cambridge Technology Inc. of Lexington, Mass. The scanning element 18 may also or instead be, but is not limited to, one or more facets of a rotating polygon, or some combination thereof.

The second optical element 20 and the third optical element 30 may also be, but are not limited to, a lens, a combination of lenses, an achromatic lens, a holographic imaging element or a mirror, an aspheric mirror or some combination thereof and may be coated with suitable anti-reflection coatings.

The scanning element 18 receives light emitted from the line shaped, incoherent light source 14 via the first optical element 16. The scanning element 18 is capable of directing the received light onto a retinal region 22 of an eye 21 via the second optical element 20 and a cornea 24 and eye lens 26 of the retinal region 22. Light reflected from the retinal region 22 is also directed back to the scanning element 18 via the second optical element 20. The reflected light received by the scanning element 18 is descanned and directed via the beam-splitter 28 and the third optical element 30 onto the detector 32.

FIG. 1B is a schematic side view of an exemplary confocal, line scanning opthalmoscope 12 of the present invention, showing the line shaped, incoherent light source 14, the first optical element 16, the scanning element 18, the second optical element 20, the beam-splitter 28, a third optical element 30 and the detector 32 from a side view. FIG. 1B also shows the eye 21 that the exemplary confocal, line scanning opthalmoscope 12 is used to image.

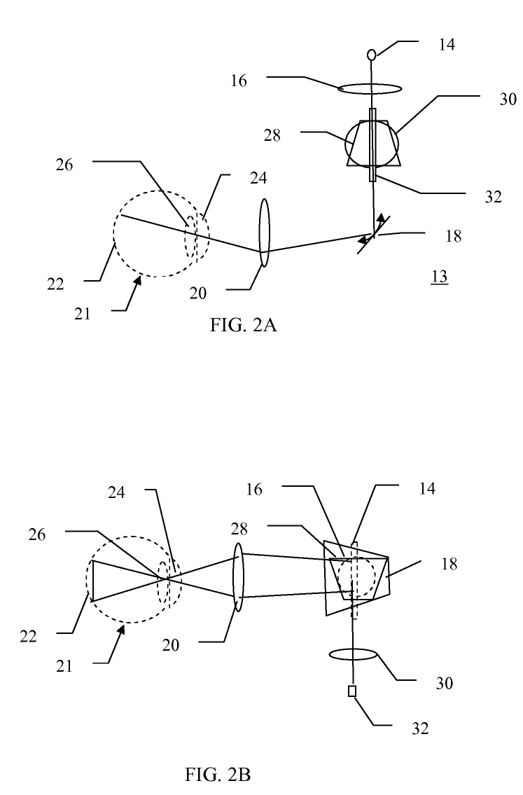

FIG. 2A is a schematic plan view of a further, exemplary confocal, line scanning opthalmoscope 13 of the present invention, showing the line shaped, incoherent light source 14, the first optical element 16, the scanning element 18, the second optical element 20, the beam-splitter 28, a third optical element 30 and the detector 32 from a side view. FIG. 2A also shows the eye 21 that the further, exemplary confocal, line scanning opthalmoscope 13 is used to image. The further, exemplary confocal, line scanning opthalmoscope 13 of FIG. 2 differs from the exemplary confocal, line scanning opthalmoscope 12 of FIG. 1 in that the beam-splitter 28 is used to deflect the optical axis from the beam-splitter 28 to the detector 32 out of the plane of the optical axis joining the line shaped, incoherent light source 14 and the scanning element 18 and the optical axis joining the scanning element 18 and the second optical element 20. The result is a more compact a further, exemplary confocal, line scanning opthalmoscope 13.

FIG. 2B is a schematic side view of a further exemplary confocal, line scanning opthalmoscope of the present invention 13, showing a side view of the compact arrangement.

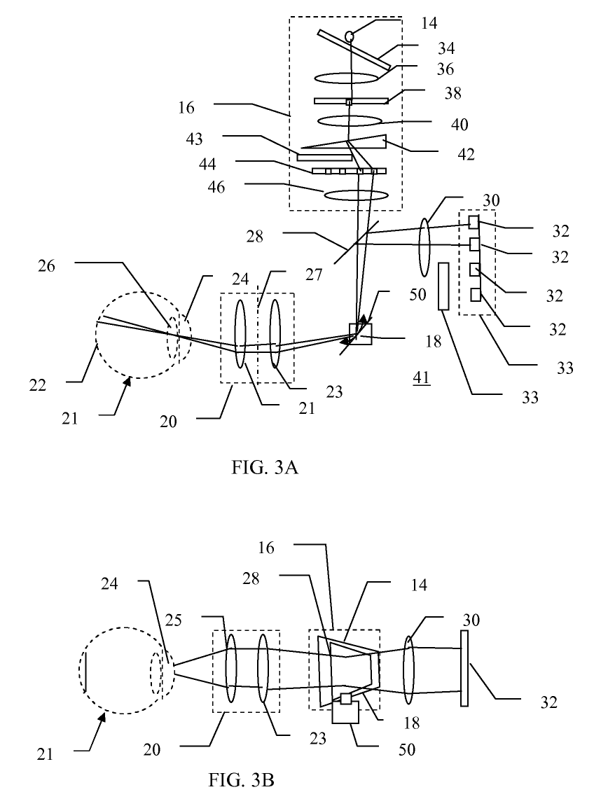

FIG. 3A is a schematic plan view of an exemplary confocal, color line scanning opthalmoscope 41 of the present invention, showing the line shaped, incoherent light source 14, the first optical element 16, the scanning element 18, the second optical element 20, the beam-splitter 28, a third optical element 30 and the detector 32 from a side view. FIG. 3A also shows the eye 21 that the exemplary confocal, color line scanning opthalmoscope 41 is used to image.

The first optical element 16 now includes a filter 34, a first slit focusing lens 36, a slit 38, a second slit focusing lens 40, a dispersive element 42, a movable filter 43, a multi-slit plate 44 and a multi-slit lens 46.

The second optical element 20 now includes two lenses, a first Badal lens 23 and a second Badal lens 25.

The line shaped, incoherent light source 14 may be, but is not limited to, a heated incandescent wire emitting light from a plurality of points along its length, an arc lamp emitting a bright white light in the gap between anode and cathode, a high brightness white light emitting diode (LED), including a custom line shaped LED, or a white light laser such as, but not limited to, a Ti-Saphire femto-second laser, or some combination thereof.

The filter 34 is selected to reflect unwanted infra-red (IR) and/or ultra-violet light and thereby prevent this unwanted light from entering the eye.

The first slit focusing lens 36 focuses the light emitted by line shaped, incoherent light source 14 onto the slit 38. The first slit focusing lens 36 may be, but is not limited to, a lens, a compound achromatic lens, a group of lenses, a mirror, a holographic lens, a fiber optic, or some combination thereof.

In a preferred embodiment, the slit 38 has a width of about 50 micrometers and a length of about 15 millimeters, though other slit configurations may also be used effectively.

The second slit focusing lens 40 focuses the light emerging from the slit 38 towards a dispersive element 42. The dispersive element 42 is used to separate the white light emerging from the slit 38 into two or more discreet wavelength bands. The dispersive element 42 may, for instance, be a prism, a diffraction grating, a holographic element or some combination thereof.

The movable filter 43 may be used to selectively transmit one or more of the portions of the electromagnetic spectrum that the dispersive element 42 has separated the white light into. For instance, in a first phase of alignment and focusing the instrument on a patent s eye, the movable filter 43 may be used to selectively transmit IR light but block visible light so as to minimize the discomfort to the patent. In a second phase, the filter 43 may be moved to allow the retina to be imaged with visible light.

The multi-slit plate 44 has two or more slits. In a preferred embodiment, the multi-slit plate 44 has four slits that may, for instance, correspond to an IR band, a red band, a green band and a blue band. In a preferred embodiment, the multi-slit plate 44 has center-to-center distances and slit lengths that correspond to the center-to-center distances and slit lengths of a multiple linear array of photo-detectors 33 after correction for possible differences in magnification by multi-slit lens 46 and the third optical element 30. In a preferred embodiment, the width of the slits in the multi-slit plate 44 are similar to the width of the individual detectors 32 in the multiple linear array of photo-detectors 33.

The slits on the multi-slit plate 44 appear as thin lines with different colors. The color and width of the spectrum transmitted by each slit is determined by the width of the slit 38 and by the properties and alignment of the dispersive element 42. The optical element 46 is used for focusing light emanating from the slits on the multi-slit plate 44 onto the scanning element 18, which in turn reflects the light to the first Badal lens 23. The second Badal lens 25 focuses the light into an image plane 27, which is optically conjugate to the plane of the retina. The slits of the multi-slit plate 44 appear in the an image plane 27 with a magnification determined by the first Badal lens 23 and the multi-slit lens 46.

The second Badal lens 25 focuses the light towards the eye pupil, and the eye focuses the four separate but parallel lines of light onto the retinal region 22. The first Badal lens 23 and the second Badal lens 25 are preferably positioned to form a ‘Badal system’ which allows for the correction of defocus errors of the eye without changing the magnification of the retinal image.

FIG. 3B is a schematic side view of an exemplary confocal, color line scanning opthalmoscope of the present invention.

The parallel lines of light which illuminate the retinal region 22 are partially reflected by the retina. The reflected light is imaged on an image plane 27 using the second Badal lens 25. The first Badal lens 23 and the second Badal lens 25 image the eye pupil onto the scanning element 18, thus light reflected by the retina and transmitted through the eye pupil, will be imaged onto the scanning element 18. In a preferred embodiment, the scanning element 18 may be a back-and-forth scanning mirror, driven by a galvanometer motor 50. The galvanometer motor 50 may, for instance be, but is not limited to, the model 6220H moving magnet closed loop galvanometer based optical scanner as supplied by Cambridge Technology Inc. of Lexington, Mass. The scanning element 18 may also or instead be, but is not limited to, one or more facets of a rotating polygon, or some combination thereof.

The beam-splitter 28 may reflect the light returning from the retinal region 22 towards the multiple linear array of photo-detectors 33. As has been described in the prior art, the beam-splitter 28 is preferably positioned in a plane which is optically conjugate to the cornea 24 so that corneal reflections may be eliminated from the retinal image.

The third optical element 30 focuses the four reflected lines of light in parallel onto the quad multiple linear array of photo-detectors 33. In a preferred embodiment, the multiple linear array of photo-detectors 33 is a single semiconductor chip containing four linear arrays of 1*1024 pixels, placed parallel to each other on the chip.

In a preferred embodiment of the exemplary confocal, color line scanning opthalmoscope 41, the multiple linear array of photo-detectors 33 comprises a quad-linear array having four linear array detectors 32, each of which is sensitive to a different part of the electromagnetic spectrum. In a preferred embodiment, the detectors 32 of the multiple linear array of photo-detector 33 are sensitive to infrared light, to red light, to green light and to blue light.

In another embodiments, detectors 32 may be sensitive to different colors, that may occur in a different order.

For instance, in a further preferred embodiment, the multiple linear array of photo-detectors 33 may be a tri-linear array such as the KODAK KLI-2113 sensor that is a high-dynamic range, multispectral, linear CCD image sensor typically used color scanner applications. In this detector, one detector 32 is sensitive to both infra red and red light, one detector 32 is sensitive to green light and one detector 32 is sensitive to blue light.

In yet another embodiment, the multiple linear array of photo-detectors 33 may comprise a 2-dimensional CMOS or CCD array, in which only selected lines may be read out and used for imaging, thereby effectively creating a multi-linear array that may be, but is not limited to, a quad-linear array or a tri-linear array.

By using appropriate electronics to read out the four linear arrays in parallel and by using appropriate electronics to synchronize array readout with the scanning mirror, four monochrome digital images of the retina may be created. Each image may be generated with a different color. The four monochrome images may be slightly shifted with respect to each other, due to the distance between the four line arrays. Using computer algorithms, this shift may be compensated and a single full color image may be created.

Moveable color filters 37 positioned before the detector, may, for instance, be used for fluorescence imaging.

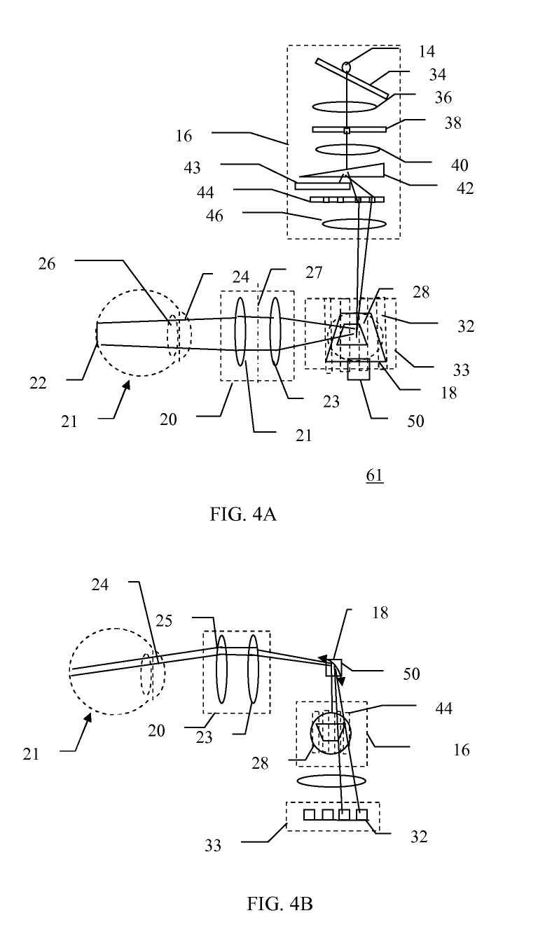

FIG. 4A is a schematic plan view of a further exemplary confocal, color line scanning opthalmoscope 61 of the present invention. The further exemplary confocal, color line scanning opthalmoscope 61 differs from the exemplary confocal, color line scanning opthalmoscope 41 of FIG. 3 in that the optical path from the line shaped, incoherent light source 14 to the beam-splitter 28 has been folded down from the plane containing the optical path from the scanning element 18 to the multiple linear array of photo-detectors 33 and the optical path from the scanning element 18 to the second optical element 20.

In this embodiment, the beam-splitter 28 may be a small turning mirror that reflects a substantial part of the illumination onto the scanning element 18, which in turn reflects the light to the first Badal lens 23. As before, the first Badal lens 23 focuses the light to the an image plane 27, which is optically conjugate to the plane of the retina. The slits of the multi-slit plate 44 appear in the an image plane 27 with a magnification determined by the first Badal lens 23 and the multi-slit lens 46.

FIG. 4B is a schematic side view of a further exemplary confocal, color line scanning opthalmoscope of the present invention.

In this embodiment of the invention, most of the light returning from the retina will pass around the beam-splitter 28 and travel towards the multiple linear array of photo-detectors 33 via third optical element 30, although some of the returning light will be reflected back to first optical element 16. As has been described in prior art, the beams-splitter 28 is preferably positioned in a plane which is optically conjugate to the cornea 24. In that case corneal reflections may be eliminated from the retinal image.

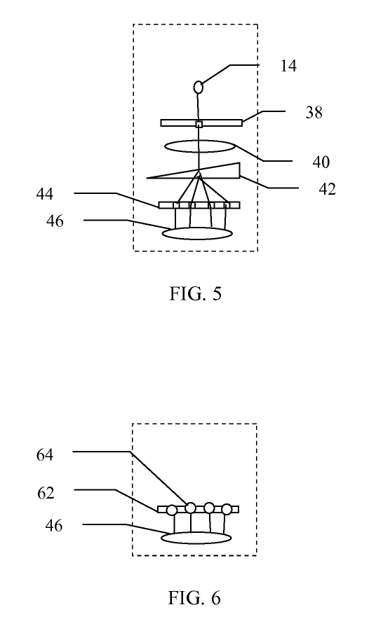

FIG. 5 shows an alternative embodiment of the illumination system, in which a straight incandescent wire or ribbon is used as source of illumination. If the width of this light source is similar to the to the width of illuminated slit 38 in the foregoing FIGS. 1, 2 and 3, there is no need for optical element 36 and slit 38, which therefore may be omitted in this embodiment.

The second slit focusing lens 40 and the dispersive element 42 ensure that light emerges from the multi-slit plate 44 as thin lines of colored light.

FIG. 6 shows an alternative embodiment of the illumination system, in which 4 parallel line-shaped LEDs as built into one component are used as source of illumination. The multi-slit plate 44 is replaced by an LED assembly 62 that may contain three or four LEDs 64, each of which may be shaped as a thin and long line. The LEDs may each have different colors.

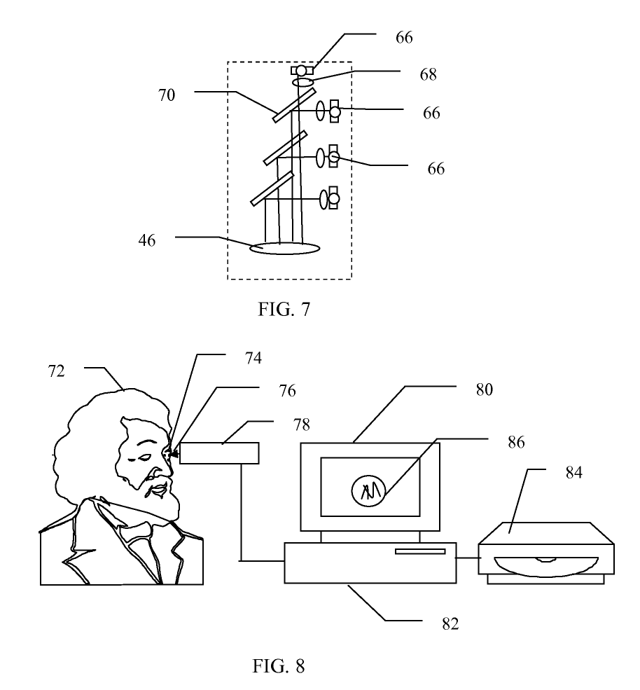

FIG. 7 shows an alternative embodiment of the illumination system, in which 4 separate-line shaped LEDs are used as source of illumination.

In a preferred embodiment, the illumination system has four custom LEDs 66, each of which may be shaped as thin, long line, and may be combined and aligned using dichroic beamsplitters 70. The lenses 68 are used for imaging the line LEDs as long, thin lines of light at an image plane 27. The LEDs may have different colors.

FIG. 8 shows a schematic view of an exemplary confocal color scanning system in use. A patient 72 is having the patent s eye 74 examined using a confocal color opthalmoscope 78. Light 76 is emitted from the confocal color opthalmoscope 78 to illuminate the retina of the patent s eye 74. Light reflected from retina of the patent s eye 74 is collected by the confocal color opthalmoscope 78. The confocal color opthalmoscope 78 may be connected to a processing unit 82. The connection to the processing unit 82 may, for instance, be via a USB connector that may also supply power to the confocal color opthalmoscope 78. The processing unit 82 may incorporate or be connected to a display 80 such as, but not limited to, a color liquid crystal display on which an image of the retina 86 may be displayed. The processing unit 82 may also or instead be connected to a data storage unit 84.

Although the invention has been described in language specific to structural features and/or methodological acts, it is to be understood that the invention defined in the appended claims is not necessarily limited to the specific features or acts described. Rather, the specific features and acts are disclosed as exemplary forms of implementing the claimed invention. Modifications may readily be devised by those ordinarily skilled in the art without departing from the spirit or scope of the present invention.

What is claimed is:

1. A confocal color opthalmoscope, comprising:
a first line shaped, incoherent light source emitting light from a plurality of points along its length; and
a scanning element that receives light emitted from said first linear, incoherent light source via a first optical element, directs said received emitted light to a second optical element that it is capable of illuminating a region of a retina of an eye with a line oriented perpendicular to a direction of scan, said second optical element also being capable of receiving light reflected from said retina of an eye and directing said received reflected light back towards said scanning element, and wherein said scanning element also descans said received reflected light and directs said descanned light via a third optical element to a first line shaped detector.

2. The opthalmoscope of claim 1, wherein said first optical element further comprises a dispersive element, a multi-slit plate and a multiple linear array of photo-detectors arranged such that each slit of said multi-slit plate is illuminated by a different spectral component of said line shaped, incoherent light source, and such that light from each of said slits of said multi-slit plate is imaged back to a different detector of said multiple linear array of photo-detectors.

3. The opthalmoscope of claim 2 in which said dispersive element is a prism, a diffraction grating or a filter.

4. The opthalmoscope of claim 2 wherein at least one of said slits of said multi-slit plate is illuminated with infra-red radiation, and at least one of said slits of said multi-slit plate is illuminated with visible light; and further comprising a movable filter capable of allowing only the infra-red light to reach said retinal region when in an initial alignment mode, thereby facilitating non-mydriatic alignment of said opthalmoscope.

5. The opthalmoscope of claim 4 wherein said second optical element comprises a first and a second Badal lens arranged in a Badal configuration, thereby enabling the correction of defocus errors of the eye without changing the magnification of the retinal image.

6. The opthalmoscope of claim 3 wherein said multi-slit plate comprises four slits, one of said four slits being illuminated with infra-red light, one with red light, one with green light and one with blue light.

7. The opthalmoscope of claim 3 wherein the combinations of wavelengths present in each slit of said multi-slit plate is selected such that a retinal image may be obtained that has colors closely resembling the colors of retinal images made with a fundus camera.

8. The opthalmoscope of claim 3 wherein the combinations of wavelengths present in each slit of said multi-slit plate is selected such that physical properties such as the concentration of oxygenated hemoglobin or the concentration of glucose may be deduced.

9. The opthalmoscope of claim 2 in which said first line shaped, incoherent light source is an incandescent strip.

10. The opthalmoscope of claim 1 wherein said first line shaped, incoherent light source is a custom line-shaped light emitting diode.

11. The opthalmoscope of claim 10 further comprising a second line-shaped, incoherent light source that is a line-shaped light emitting diode, having a different spectral output from said first line shaped light source and arranged to be substantially coplanar and substantially parallel to said first line shaped light source.

12. The opthalmoscope of claim 11 wherein said first line shaped, incoherent light source emits infra-red radiation, and second line-shaped, incoherent light source emits visible light; and further comprising electronic means for selectively turning on said light sources or a movable filter capable of allowing only the infra-red light to reach said retinal region when in an initial alignment mode, thereby facilitating non-mydriatic alignment of said opthalmoscope.

13. The opthalmoscope of claim 11 wherein the combinations of wavelengths present in said first and second line-shaped, incoherent light sources are selected such that a retinal image may be obtained that has colors closely resembling the colors of retinal images made with a fundus camera.

14. The opthalmoscope of claim 11 wherein the combinations of wavelengths present in said first and second line-shaped, incoherent light sources are selected such that physical properties such as the concentration of oxygenated hemoglobin or the concentration of glucose may be deduced.

15. The opthalmoscope of claim 10 further comprising a second line-shaped, incoherent light source that is a line-shaped light emitting diode, having a different spectral output from said first line shaped light source; and a first dichroic beamsplitter arranged to allow said first and second incoherent line sources to be focused to a common plane.

16. The opthalmoscope of claim 15 wherein said first line shaped, incoherent light source emits infra-red radiation, and second line-shaped, incoherent light source emits visible light; and further comprising a movable filter capable of allowing only the infra-red light to reach said retinal region when in an initial alignment mode, thereby facilitating non-mydriatic alignment of said opthalmoscope.

17. The opthalmoscope of claim 15 wherein the combinations of wavelengths present in said first and second line-shaped, incoherent light sources are selected such that a retinal image may be obtained that has colors closely resembling the colors of retinal images made with a fundus camera.

18. The opthalmoscope of claim 15 wherein the combinations of wavelengths present in said first and second line-shaped, incoherent light sources are selected such that physical properties such as the concentration of oxygenated hemoglobin or the concentration of glucose may be deduced.
